(19) United States
(12) Patent Application Publication
(10) Pub. No.: US 2026/0284083 A1
(43) Pub. Date: Sep. 24, 2026

(54) PROPHYLAXIS AND TREATMENT OF CORONAVIRUS INFECTION

(71) Applicants: Ludwig ENGLMEIER, Oberaudorf (DE); Bourgelat Pty Ltd, Victoria (AU)

(72) Inventor: Ludwig ENGLMEIER, Oberaudorf (DE)

(21) Appl. No.: 19/573,179

(22) Filed: Mar. 20, 2026

Related U.S. Application Data
(63) Continuation of application No. 17/919,499, filed on Apr. 17, 2021, now abandoned.

(30) Foreign Application Priority Data
Apr. 19, 2020 (EP) 20000159.2
Apr. 21, 2020 (EP) 20000163.4
May 10, 2020 (EP) 20173791.3
May 12, 2020 (EP) 20174062.8
May 12, 2020 (EP) 20174152.7
May 13, 2020 (EP) 20174383.8

(51) Int. Cl. A61K 31/593; A61K 31/4706; A61K 31/473; A61P 31/14

(52) U.S. Cl. CPC A61K 31/593; A61K 31/4706; A61K 31/473; A61P 31/14

(57) ABSTRACT

The present invention relates to the use of antagonists of tlr7-signalling for the treatment of COVID-19 patients, wherein the antagonists of tlr7-signalling are administered only after serovonversion. The invention also relates to a tlr agonist for use in the one-time prophylactic treatment of a subpopulation against an infection with a coronavirus, wherein the subpopulation is a collection of asymptomatic subjects in proximity to subjects of the population who show symptoms of infection.

### FIELD OF THE INVENTION

The present invention relates to the use of antagonists of tlr7-signalling for the treatment of COVID-19 patients, wherein the antagonists of tlr7-signalling are administered only after serovonversion. The invention also relates to a tlr agonist for use in the one-time prophylactic treatment of a subpopulation against an infection with a coronavirus, wherein the subpopulation is a collection of asymptomatic subjects in proximity to subjects of the population who show symptoms of infection.

### BACKGROUND OF THE INVENTION

In December 2019, health authorities in Wuhan, China, identified a cluster of pneumonia cases of unknown aetiology linked to the city's South China Seafood Market.

Subsequent investigations revealed a novel coronavirus, SARS-COV-2, as the causative agent now at the heart of a major outbreak.

The clinical disease termed COVID-19 is caused by a novel betacoronavirus, now named SARS-COV-2. SARS-COV-2 shares 79% sequence identity with SARS-COV, the virus which caused a major outbreak in 2002-2003. In common with SARS-COV, SARS-COV-2 utilises the ACE-2 receptor for cell entry. The most common symptoms being reported are fever, cough or chest tightness, and dyspnoea. Most cases are reported to experience a mild illness course (Lake, MA; Clin. Med. (Lond) 2020 March; 20(2): 124-127), however, as of the priority date of this patent application, already about 150000 people have died from COVID-19 (https://www.worldometers.info/coronavirus/ on 17 Apr. 2020).

In the absence of immunity against the new virus, infection with SARS-COV-2 can spread exponentially in a population. This spread has been checked and contained in several countries, but harsh measures such as a “lockdown” or quarantaine-like mobility restrictions to people have been necessary to achieve this result.

But parts of the general population cannot stay at home because their work is essential for even a basic functioning of society. In particular medical personnel cannot just “stay at home”, but is even exposed to a high risk of contracting the virus by caring for COVID-19 patients. Many doctors and nurses get infected with the virus despite the implementation of vigorous hygienic measures to protect against virus exposure.

It has been common sense that overcoming this disease would require the successful development of a vaccine against SARS-COV-2. However, vaccine development against the closely related SARS-COV-1 virus and against MERS has been so far unsuccessful, despite years of research. There is thus an urgent need for providing immunity against SARS-COV-2 or future pandemic viruses.

It is of course most desirable to achieve immunization of a collection of individuals by a vaccination with minimal side effects which has undergone vigorous testing and clinical trials.

Alas, this is often not available. In such a situation immunization of a collection of individuals—a subpopulation—among a larger population would only achievable if the subpopulation contracted the disease, with all dire consequences that this might have.

In the context of the current COVID-19 pandemic it has indeed been suggested that subpopulations having an infection fatality rate that is known to be or expected to be below the average infection fatality rate of the population, such as young adults up to the age of 40 years, should be allowed to contract the virus. And politicians might even decide that this will be done in the near future.

Of course, even though the fatality rate is low in the above-mentioned patient group, it is not zero, so some of the exposed young people would die from the exposure and a substantial fraction would come down with serious disease symptoms.

In seriously ill COVID-19 patients pathological changes like thrombosis, embolism, involvement of the cardiovascular tract, kidney failure, loss of smell and skin lesions have been observed. Only few drugs, like dexamethasone, show a significant effect in the treatment of the “cytokine storm” of severe COVID cases. Other drugs, like remdesivir and hydroxychloroquine, have been used in the early stages of the disease due to their antiviral activity. However, the significance of any positive effect of their use is a matter of dispute and discussion. There is therefore an urgent need for drugs and treatment regimens that help COVID patients.

### SUMMARY OF THE INVENTION

In the present Codiv-19 pandemia several groups apparently have a high risk of developing severe COVID-19: obese people, people of old age, men and patients suffering from systemic lupus erythematosus. The present inventor has identified a common denominator for these groups: low tlr7 activity either by low gene dosage, as in men (tlr7 escapes X-chromosome inactivation in women, who thus have a higher expression of tlr7 than men, or low tlr7 activity due to toll-like receptor tolerance in obese, old and SLE patients. Obese patients show a chronic activation of tlr7 (Revelo et al., Cell Rep. 2016 July 19; 16(3):717-30) which leads to the development of tolerance to tlr7 signalling over time (Michaelis et al., Brain Behav Immun. 2019 Nov; 82:338-353). People of old age overexpress miR-146a, leading to a constant stimulation of tlrs and consequently also to the development of tlr tolerance. SLE patients suffer from an excess of tlr7 substrates produced by the body. Thus, it can be expected that also in this case the body will try to downregulate the sensitivity of toll-like receptor signalling. When a viral infection occurs in a situation of developed tlr tolerance, the initial response of the body to the infection will be delayed or insufficient—due to the downregulated sensitivity of tlrs—thus exposing the affected individuals to severe disease. These observations point towards tlr agonists as being of potential help in the Covid-19 pandemic, as will be explained below.

It is a further prediction of the current invention that once a virus infection, such as infection with SARS-COV-2, has established itself in an at-risk patient (the obese and elderly in the case of SARS-COV-2)—due to an initially laggard antiviral innate immune response as a consequence of tlr7 desensitization—the tlr7-signal from the viral RNA will at one point become strong enough to even be sensed by the ineffective innate immune system of the at-risk patient. The body will respond by reverting desensitization of the tlr7-signalling pathway, which will regain its normal sensitivity so that the innate immune system is then in a better state to fight the virus. It is a prediction of the present invention that this will put at-risk patients also at a disadvantage at a later stage of the infection due to then overactive tlr7-signalling.

The present invention therefore relates to an antagonist of tlr7-signalling for use in the treatment of a disease caused by a coronavirus, such as COVID-19 (caused by SARS-COV-2), wherein the antagonist of tlr7-signalling is to be administered after seroconversion of the coronavirus patient. Tlr7-signalling is important for linking the innate immune response with the development of the adaptive response. The present inventor reasons that tlr7-signalling should only be inhibited once this job of tlr-signalling has been done so that antibodies are being produced and viral clearance can proceed.

The present invention also relates to a method of providing immunity against a virus to a selected subpopulation of a larger population by administering an agonist of a toll-like-receptor (tlr) to individuals from the selected subpopulation in a first step, and then exposing the individuals who had been pretreated with the tlr agonist to the virus.

Without wishing to be bound to any theory, the pretreatment of the subpopulation with the tlr agonist will boost the innate immune system of the pretreated individuals, which in turn will help them to better cope with the virus as long as the immune-boost by the tlr agonist is still active. Exposure to the virus will then lead to an infection, which is, however, less severe, but still leads to an immune response in the individuals of the subpopulation and to subsequent immunization of the subpopulation.

Zhao et al., J. Virol. 2012 Nov, 86(21):11416-11424 have shown that intranasal treatment with poly(I:C) protects aged mice from lethal respiratory virus infections. They suggest poly(I:C) for further investigation for prophylaxis in aged populations at high risk.

However, they do not suggest, after treatment of subjects with poly(I:C), to actually expose pretreated subjects to the virus for the purpose of creating immunity in the subjects. However, the results from Zhao et al. make it plausible that a pretreatment of a subpopulation with a tlr agonist, and in particular pretreatment of a subpopulation that is less vulnerable to the effects of the virus infection, would convey some protection to the pretreated subpopulation upon exposure to the virus in the period after the pretreatment when the immune-system is still boosted.

In the context of the present SARS-Cov-2 pandemic, the present invention will lower the already very low infection fatality rate and the low rate of severe COVID-19 in young adults below the age of 40 even further. While it will remain a difficult political decision, whether the remaining lowered fatality rate and rate of severe disease are acceptable from an ethical standpoint, the present invention at least provides a less undesirable option for creating immunity in a subpopulation than virus infection without pretreatment, for example in so-called “Corona parties”.

The present invention also relates to the use of a toll-like receptor agonist in the immunization of a subpopulation of a population during a virus outbreak.

The present invention also relates to a one time administration of a tlr agonist for the immediate prophylactic treatment of a virus infection in a subpopulation, wherein the subpopulation is a collection of asymptomatic subjects in proximity to subjects of the population who show symptoms of virus infection.

### DETAILED DESCRIPTION OF THE INVENTION

The present invention relates to a method of providing immunity against a virus to a selected subpopulation of a larger population by
a) administering an agonist of a toll-like-receptor to individuals from the selected subpopulation, and then
b) exposing the individuals from step a) to the virus.

The administration of the toll-like receptor agonist shortly prior to the exposure to the virus activates the innate immune system of the subjects of the subpopulation and enables them to better deal with the virus once exposed to it in the period when the immune system is still boosted by the effect of the tlr agonist. The time between administration of the tlr agonist and exposure to the virus can vary for the tlr agonist to be used and is preferably at most seven days, more preferably at most 5 days and most preferably exposure to the virus is effected in the period from immediately after administration of the tlr agonist to three days after administration of the tlr agonist, such as from one hour to 3 days after administration of the tlr agonist.

“Population” means a group of individual organisms of the same species that live in the same geographic area. For example all human beings living in the United States of America form the “population” of the USA. Or all cows of a farm form the population of cows on the farm.

“Subpopulation” means a proper subset of the population, wherein the individuals from the subset share at least one characteristic with one another that is not shared by all individuals of the population. For example, old people above the age of 60 in the USA would form a subpopulation within the larger population of the USA.

While the method of the present invention would be predicted to work for all organisms that have toll-like-receptors as part of their immune system and that have both an innate and an adaptive immune system, preferred populations are vertebrate populations and in particular mammalian populations. Preferred populations are human populations, and populations of farm animals, such as cow, pig, horse, goat or sheep.

Typically a subpopulation is chosen for immunization that has an infection fatality rate that is known to be or expected to be below the average infection fatality rate of the population. In the current Covid-19 epidemic, for example, it is known that children from age 2 to young adults to age 30 apparently have a lower risk to die from SARS-COV-2 infection than old people above the age of 60. It has therefore been discussed to infect this group on purpose so as to generate a higher rate of immune people in the population and to eventually protect the older people via “herd immunity”. However, it is felt that the mortality and in particular the rate of severe disease would still be too high for such an approach. The method of the present invention would make such an approach more viable as it would further decrease the already low mortality rate.

Another interesting subpopulation for the method of the present invention is a subpopulation that is at an increased risk of exposure to the virus, such as medical personnel.

The numbers of infected doctors and nurses, who care for Covid-19-patients, are too high. In particular in situations where protective personal equipment is on short supply and where an increased rate of infection among medical personnel is to be, unfortunately, expected, it might be desirable to immunize this subpopulation before expected virus exposure.

While the method of the present invention is predicted to work against infections by all pathogens that can be recognized by the innate immune system, preferred pathogens to which immunity is to be conferred by the method of the present invention are viruses, and in particular—if only under the impression of the present pandemic—it is a virus where the virus causes a respiratory disease. The virus can be an RNA-virus, such as a coronavirus, and in particular a SARS-like virus like SARS-COV-2.

Of course, if the population to be immunized changes, for example if a population of swine is to be immunized, also the virus changes correspondingly, for example for swine it might become desirable to immunize against against classical swine fever, caused by a pestivirus, or against African swine fever, caused by an asfarvirus.

While the method of the present invention is predicted to work also if the tlr agonist is administered systemically and later exposure to the virus is tissue-specific, it is preferred that exposure to the virus in step b) and administration of the toll-like-receptor agonist in step a) are effected to the same part of the body. For example, the part of the body can be the upper respiratory tract, such as the nasal cavity, which is a particularly desired combination of tlr agonist administration and virus exposure for respiratory viruses. For example, to be seen in the context of the current Covid-19 pandemic, the tlr agonist would first be administered to the nasal cavity (for example GSK2245035 is a tlr7 agonist in clinical trials for intranasal administration) and then shortly thereafter the SARS-COV-2 virus would be administered to the nasal cavity as well.

Most toll-like receptors signal via MyD88 (or a mammalian homologue thereof) and activate the gene expression of antiviral cytokines and chemokines. For the method of the present invention, the nature of the tlr agonist can be selected so that it activates those toll like receptors that are involved in the individual's innate immune response against the pathogen against which immunization is to be effected. For example, when the population is a human population and the pathogen is a virus, the toll-like receptors tlr3, tlr7, tlr8 and tlr9 are known to be involved in the recognition of viral antigens.

The present invention can relate to a method, where the toll-like receptor agonist in step a) is selected to activate a toll-like receptor that is not involved in the innate immune response against the pathogen of step b).

But the present invention also relates to a method, where the toll-like receptor agonist in step a) is selected to activate a toll-like receptor that is involved in the innate immune response against the pathogen of step b). In the preferred situation of providing immunization against a virus, preferred tlr agonists are a tlr3 agonist, a tlr4 agonist, a tlr7 agonist, a tlr8 agonist and/or a tlr9 agonist. The invention also relates to a method wherein the tlr agonist is selected from the following groups: tlr3, tlr4 and tlr9; tlr3, tlr4, tlr7 and tlr8; tlr4, tlr7, tlr8 and tlr9; tlr7, tlr8 and tlr9; tlr3, tlr7 and tlr8; tlr4, tlr7 and tlr9.

Many tlr agonists have been described in the literature and formulations for systemic administration but also local administration have been described and sometimes also been developed to the stage of being tested in clinical trials.

A preferred tlr agonist is Poly(I:C). Polyinosinic:polycytidylic acid is a mismatched double-stranded RNA with one strand being a polymer of inosinic acid, the other strand a polymer of cytidilic acid. It is a strong agonist of tlr3 and is a component of approved vaccines. Formulations of poly(I:C) have been described in the literature. For example AU2014345667A1 describes a formulation for administration to the upper respiratory tract. Malcolm et al. (Antiviral Res. 2018 May; 153:70-77) describe a nasal powder comprising poly(I:C)-PrEP-001 and dosages and modes of administration thereof, which can be used in the present invention.

Another preferred tlr agonist is R848 (resiquimod). Resiquimod is a dual tlr7/8 agonist. Formulations of resiquimod have been described in the literature. For example WO2017/004421A1 describes topical and injectable formulations (the injectable formulations could be useful for systemic administration of resiquimod) and US2004/136917A1 describes formulations for administration to the upper respiratory tract.

Imiquimod is an FDA-approved tlr7 agonist. Formulations of imiquimod have been described in the literature. US2004/136917A1 describes, for example, formulations for administration to the upper respiratory tract.

VTX-2337 (motolimod) is a tlr8 agonist and has been used in several clinical phase II studies for the treatment of various cancer types. In the “Active8” clinical trial a subcutaneous formulation of motolimod was used, but other formulations for systemic administration or administration to the upper respiratory tract are described, for example WO2017/079283A1 describes dosages and formulation.

GLA-SE (glucopyranosyl lipid-A (GLA) in a stable, oil-in-water emulsion) is a tlr4 agonist and has been used as an adjuvant in numerous vaccines. Formulations comprising GLA-SE have been described in the literature. For example WO2015/112485A1 describes formulations and dosages of GLA-SE in combination with an antigen. The skilled person will appreciate that GLA-SE formulations suitable in the methods of the present invention can simply be made by taking a described vaccine formulation comprising GLA-SE and omitting the respective vaccine antigen. In an urgency situation, however, the existing vaccines might even be useful.

SD-101 is a tlr9 agonist-a synthetic CpG oligonucleotide, and has been used in clinical trials (for example see Clinical Trials. gov identifier NCT02521870, where it has been administered by injection).

GSK 2245035 (for structural information see Biggadike et al. in J. Med. Chem. 2016, 59, 5, 1711-1726) is a tlr7 agonist and has been used in clinical trials as a nasal spray solution (see, for example ClinicalTrials. gov Identifier: NCT02833974).

RO7119929 is a tlr7 agonist being tested by Roche in a clinical trial against liver cancer, where it is given orally (see, for example, ClinicalTrials. gov Identifier: NCT04338685).

WO2015162075A1 describes further tlr7 agonists by Roche.

GSK1795091 is a tlr4 agonist and has been tested in clinical dose-finding trials, where it was administered systemically by intravenous injection (see, for example ClinicalTrials.gov Identifier: NCT02798978).

The present invention also relates to the use of a toll-like receptor agonist in any one of the methods of providing immunity against a virus to a selected subpopulation of a larger population. The tlr receptors, mode of administration, viruses and other variations of the methods can be as described above, in particular as described above for the preferred embodiments.

The present invention also relates to the use of a toll-like receptor agonist for the immunization of a subpopulation of a population during a virus outbreak. The virus outbreak can be a virus epidemic or a pandemic, as in the current example of the SARS-COV-2 pandemic.

The tlr receptors, mode of administration, viruses and other variations of the methods can be as described above, in particular as described above for the preferred embodiments.

In particular, the present invention relates to the use of a toll-like receptor agonist for the immunization of a subpopulation of a population during a virus outbreak, wherein the toll-like receptor agonist is to be administered to a subpopulation who is at risk of being exposed to the virus within seven days after administration of the toll-like receptor agonist.

In a further embodiment, the present invention relates to a tlr agonist for use in the one-time prophylactic treatment against an infection, such as a virus infection, of a subpopulation, wherein the subpopulation is a collection of asymptomatic subjects in proximity to subjects of the population who show symptoms of infection, in particular virus infection.

Tlr agonists have been described for the prophylactic treatment of virus infections (see, for example, Zhao et al., J. Virol. 2012 Nov, 86(21):11416-11424. Also the tlr agonist Bacillus Calmette-Guérin vaccine has been discussed for Covid-19 prevention.

However, the authors who have suggested tlr agonists for prophylactic treatment fail to appreciate that i) the stimulation of the innate immune system by administration of the tlr agonist only lasts for a short period of time, and/or ii) repeated administration of the tlr agonist leads to a phenomenon called “tlr tolerance”, where the body decreases the strength of the antiviral response in reaction to repeated administration of a tlr agonist further and further until the organism might become even more vulnerable to an infection after repeated doses of tlr agonist administration.

In other words, and explained for the example of SARS-COV-2, administration of the BCG “vaccine” would only have a beneficial effect in the first week after its administration, while after week 1 a protective effect would not be seen, and also not the lasting effect of a “vaccination”.

A “one time administration” is administration of the tlr agonist on only one or two subsequent days. This should lead to a single peak of the tlr agonist plasma concentration followed by a complete absence of the tlr agonist in the plasma and at least two more weeks thereafter, where the tlr agonist is not administered. This is in contrast to dosage regimens where administration of the tlr agonist is repeated to keep the plasma level of the tlr agonist at a level above the detection limit.

A preferred example of a one-time administration is where the tlr agonist is administered only within a period of 24 hours, such as within a period of 12 hours or more preferably within a period of 6 hours, 3 hours, 2 hours, 1 hour or 30 minutes. It is most convenient to only administer a single dose of the tlr agonist, such as the tlr agonist in a single tablet, a single injection or a single nasal spray inhalation.

Preferred tlr agonists are a tlr3 agonist, a tlr4 agonist, a tlr7 agonist, a tlr8 agonist and/or a tlr9 agonist. The invention also relates to a tlr agonist for the one-time administration of the present invention, wherein the tlr agonist is selected from the following groups: tlr3, tlr4 and tlr9; tlr3, tlr4, tlr7 and tlr8; tlr4, tlr7, tlr8 and tlr9; tlr7, tlr8 and tlr9; tlr3, tlr7 and tlr8; tlr4, tlr7 and tlr9.

Preferred examples of tlr agonists are selected from the group consisting of polyinosinic: polycytidylic acid, resiquimod, imiquimod, motolimod, GLA-SE, SD-101, GSK2245035, RO7119929, and GSK1795091.

The invention also relates to tlr agonists for the one-time administration of the present invention, wherein the tlr agonist is not polyinosinic:polycytidylic acid. The invention also relates to tlr agonists for the one-time administration of the present invention, wherein the tlr agonist is not resiquimod. The invention also relates to tlr agonists for the one-time administration of the present invention, wherein the tlr agonist is not imiquimod. The invention also relates to tlr agonists for the one-time administration of the present invention, wherein the tlr agonist is not motolimod. The invention also relates to tlr agonists for the one-time administration of the present invention, wherein the tlr agonist is not GLA-SE. The invention also relates to tlr agonists for the one-time administration of the present invention, wherein the tlr agonist is not GSK2245035. The invention also relates to tlr agonists for the one-time administration of the present invention, wherein the tlr agonist is not RO7119929. The invention also relates to tlr agonists for the one-time administration of the present invention, wherein the tlr agonist is not GSK1795091.

The invention also relates to tlr agonists for the one-time administration of the present invention, wherein the virus causes a respiratory disease, such as an RNA-virus, such as a coronavirus, and in particular a SARS-like virus like SARS-COV-2.

Other preferred viruses are a pestivirus, or an asfarvirus.

The one-time prophylactic treatment of the present invention can be seen as an emergency salvage measure of a group at immediate risk of contracting the infectious agent, such as an infectious bacteria or an infective virus, for example a coronavirus like SARS-COV-2.

In the case where the population is a human population, such a situation is, for example, when medical personnel of a hospital is about to receive subjects who show symptoms of virus infection, in particular when personal protective equipment is limited or not available. The medical personnel would be at an extremely high risk of contracting the virus in such a situation and would benefit from a tlr agonist, which can boost their immune system during the short time, when the first viral exposure can be expected.

Another emergency situation, where the one time prophylactic treatment of the present invention would help, is a situation where a vulnerable subpopulation, a subpopulation having an infection fatality rate that is known to be or expected to be above the average infection fatality rate of the population, is at immediate risk of contracting the virus. Taking the Covid-19 pandemic as an example, this is the case, when a first person with possible symptoms of Covid-19 is detected in an old people's home. In such a situation, the tlr agonist should be administered to the subpopulation of asymptomatic individuals. In such a situation it must be of concern that these vulnerable individuals might get exposed to the virus in the next minutes of hours, and in such a situation boosting the innate immune system of still asymptomatic individuals is expected to reduce the number of patients who develop severe Covid-19 and who die.

In the case where the population of vertebrates, such as a mammalian animal population, and for example farm animals like pigs, cows, sheep, goats or horses, such an emergency situation can also arise. This will be explained by following example of swine fever, which is a highly lethal viral disease of pigs caused by a flavivirus (in the case of classical swine fever) or an asfarvirus (in the case of African swine fever). It is current practice to cull the complete swine population of a farm where one animal with swine fever has been detected, as the disease is highly contagious among swine and as there is no treatment for the disease. The tlr agonists for the one-time prophylactic treatment of the present invention present invention would allow a different approach when a first pig with possible symptoms of swine fever is detected in a farm. In such a situation, the tlr agonist should be administered to the subpopulation of asymptomatic pigs. In such a situation it must be of concern that these vulnerable pigs would get exposed to the virus in the next minutes of hours, and in such a situation boosting the innate immune system of still asymptomatic pigs is expected to reduce the number of pigs who die from swine fever. This would have the advantage that a part of the swine population would survive the swine fever and recover from it, resulting to a lesser economic loss to the farmer than a culling of the whole population of swine on his farm.

It is a prediction of the current invention that once a virus infection, such as infection with SARS-COV-2, has established itself in an at-risk patient (the obese and elderly in the case of SARS-COV-2)—due to an initially laggard antiviral innate immune response as a consequence of tlr7 desensitization—the tlr7-signal from the viral RNA will at one point become strong enough to even be sensed by the ineffective innate immune system of the at-risk patient. The body will respond by reverting desensitization of the tlr7-receptor, which will regain its normal sensitivity so that the innate immune system is then in a better state to fight the virus. It is a prediction of the present invention that this will put at-risk patients also at a disadvantage at a later stage of the infection.

Experiments in untreated lupus-prone mice are in line with the proposed model. Lupus-prone MRL/MpJ-Fas(Ipr) mice did on the one hand show accelerated viral clearance upon influenza infection (Slight-Webb SR et al., J Autoimmun. 2015 February; 57:66-76.), and thus demonstrated a beneficial role of high tlr7-activity in the initial phase of virus infection. But the same mice later showed a severe disadvantage of high tlr7-activity after viral clearance, since the lupus-prone mice had severe complications during the contraction and resolution phase of the infection with widespread pulmonary inflammation. This points to a positive effect of high tlr7-activity during the early stages of infection—further supporting the ideas of the present invention about the feasibility of prophylactic treatments with tlr-agonists—but also to a negative effect of high tlr7 activity during the late stages of an infection (the lupus-prone mice have a high tlr7 activity throughout the experiment, in line with being a model for lupus).

What is the underlying reason for the negative effect of high tlr7-activity during the late stage of an infection, even after the virus has been cleared? It may be that the continued tlr7-activity in the lupus-prone mice after virus clearance prevents their immune system from realizing that the virus is indeed gone. Thus, the immune system of those mice doesn't realize that time has come to contract and resolve infection.

If the innate immune system of obese people and elderly people reacted to the establishment of a severe SARS-COV-2 infection (due to an initially laggard tlr7-response as suggested herein) by restoring normal sensitivity of tlr7 after the initially delayed recognition of the viral infection, then they would find themselves in a situation which is similar to the lupus-prone mice of Slight-Webb et al.: at a late stage of infection tlr7-signalling caused by the chronic intrinsic tlr7-substrates to a tlr7 receptor, which has regained normal sensitivity, would continue even after virus clearance and the innate immune system would effectively continue fighting a virus that is no longer there.

Even before viral clearance, overall tlr7-signalling in at-risk patients would be greatly increased compared to young, non-obese patients without intrinsic tlr7-substrates because the desensitized tlr7 receptor would not only signal to single stranded viral RNA, but also to the intrinsic tlr7-substrates in the obese and elderly. Thus, obese and elderly patients would be predicted to suffer from a lupus-like state caused by an abundance of tlr7-substrates in the body—form the virus AND from the intrinsic tlr7-substrates (which had caused the desensitized state of tlr7 prior to infection by chronic activation of tlr7).

It is interesting to note that high tlr7 activity has been implied in triggering blood-clotting during sepsis. Thus, there might also be a link between tlr7 and the pathological changes—thrombosis, embolism—which can be observed in the seriously ill COVID-19 patients. It is also interesting that vitamin D3, which has recently been described to protect severely ill COVID-19 patients from thrombosis and embolism, has been also described as down-regulating tlr7 gene expression in SLE-patients.

Moreover, the pathological changes seen in some COVID-19 patients, such as an involvement of the cardiovascular tract, kidney failure and skin lesions, are also pathologies described for SLE patients. Even the peculiar loss of smell which has been described in some COVID-19 patients is common between COVID-19 patients and SLE patients! Taking all of this together, severely ill COVID-19 patients at a later stage of infection, such as >10 days post infection, suffer from a SLE-like disease state, probably caused by excessive tlr7-signalling.

Thus, treatments that help to alleviate symptoms of SLE should alleviate symptoms of severe COVID-19 as well. For the use of hydroxychloroquine, this model would predict that it should not be used as an antiviral agent in the early stages of an infection—where any positive effects on viral replication would by counteracted by its interference with a proper innate antiviral immune response—but rather it should be used to suppress the negative effects of tlr7-signalling during the contraction and resolution phase of the infection and thus with a mechanistic logic that is very similar to its use in SLE patients. Thus—and contrary to the current use of hydroxychloroquine at an early stage of the infection, where it will interfere with tlr7's important role to stimulate innate immunity and to initiate seroconversion-it is important to wait until seroconversion before using an antagonist of tlr7-signalling in the treatment of a severe coronavirus disease like COVID-19.

The present invention therefore also relates to an antagonist of tlr7-signalling for use in the treatment of pulmonary inflammation resulting from viral infection, in particular wherein the antagonist of tlr7-signalling is to be administered after viral clearance.

The present invention therefore also relates to an antagonist of tlr7-signalling for use in the treatment of a disease caused by a coronavirus, such as COVID-19 (caused by SARS-COV-2), wherein the antagonist of tlr7-signalling is to be administered after seroconversion of the coronavirus patient. For example a COVID-19 patient who has developed antibodies specific for SARS-COV-2 is a preferred patient.

“Seroconversion” within the meaning of the present invention means that specific antibodies against SARS-COV-2 have developed and become detectable in the serum of COVID-19 patients. Numerous antibody tests specific for antibodies against SARS-COV-2 have been developed by now, but the skilled person will appreciate that these tests are of variable sensitivity and/or specificity and will therefore choose a thoroughly validated antibody test, and will preferably use two independent tests, to determine seroconversion in a COVID-19 patient. Preferred antibody tests are tests which have been reviewed by the US food and drug administration and which have received at least an Emergency Use Authorization, preferably which have received a proper use authorization. Tests for seroconversion should be carried out by a laboratory certified under CLIA (the FDAs Clinical Laboratory Improvement Amendments) to perform high complexity testing. In non-US jurisdictions the skilled person would turn to laboratories and tests which fulfill equivalent high quality standards.

The “antagonist of tlr7-signalling” is a molecule that prevents or reduces NFKB-activated gene expression in response to tlr7-activation by single stranded viral RNA at an effective concentration that is safe for administration to a human being. Some antagonist of tlr7-signalling are small molecules that can bind tlr7 directly and inhibit it. Other antagonists of tlr7-signalling, like chloroquine and hydroxychloroquine, can inhibit tlr7-signalling indirectly, for example by interfering with proper tlr7-processing or by interfering with delivery of single stranded virus RNA to the endosome, where tlr7 would normally bind ss viral RNA and trigger NFKB-activation via activation of MyD88. Also molecules which interfere with the downstream signalling pathway between MyD88 and NFKB are antagonists of tlr7-signalling within the meaning of the present invention. Also vitamin D3, which decreases tlr7 gene expression in SLE-patients, antagonizes tlr7 signalling.

“Viral clearance” within the meaning of the present invention means that the body has eliminated the virus. This is typically determined by PCR-or RT-PCR based assays, which are highly sensitive for the presence of the genetic material of the virus. The skilled person will know, which samples from a patient need to be taken and analyzed in order to determine viral clearance. In the case of SARS-COV-2 for example, samples from stool and from lung-derived sputum show a longer presence of the virus than samples taken from the upper respiratory tract. The skilled person will therefore verify “viral clearance” preferably by at least to negative results in (RT-)PCR-analysis of patient samples, which have been taken from locations in the body where the virus is known to persist the longest, preferably taken from at least two independent locations.

Preferred antagonists of tlr7-signalling are molecules that are approved for medicinal use in human beings, such as chloroquine and hydroxychloroquine. Effective concentrations, dosages and dosage regimens are well known for these approved drugs, and the dosages and dosage regimens used for the treatment of SLE, as described in the product leaflets for Plaquenil® and Aralen®, can be used in the treatment of pulmonary inflammation of the present invention, with the proviso that they are to be administered after viral clearance. They are to be administered until the pathological signs of pulmonary inflammation have improved.

COVID-19 patients often die from thrombosis and/or lung embolism. Tlr7 has been implicated in the generation of these pathologies. As a consequence of antagonizing detrimental tlr7-signalling after viral clearance, the antagonists of tlr7-signalling should also improve thrombosis and/or protect from lung embolism. The present invention therefore also relates to an antagonist of tlr7-signalling for use in the treatment of thrombosis and/or the prevention of lung embolism resulting from viral infection, in particular wherein the antagonist of tlr7-signalling is to be administered after viral clearance and wherein the virus is a coronavirus, such as SARS-COV-1 or SARS-COV-2.

Venous thromboembolism is a condition in which a blood clot forms most often in the deep veins of the leg, groin or arm (known as deep vein thrombosis, DVT) and travels in the circulation. If such a blood clot lodges in the lungs, it can cause pulmonary embolism.

Kidney injury as used herein is defined as a 2.0 fold increase in sCr (serum creatinine) or a >50% decrease in the glomerular filtration rate, in line with the RIFLE criteria for staging acute kidney injury.

Cytokine release syndrome within the meaning of the present invention is at least grade 2 CRS according to the revised grading system described in “Current concepts in the diagnosis and management of cytokine release syndrome”, Blood (2014) 124(2):188-195 by Lee et al.

Neurological symptoms described for COVID-19 are acute cerebrovascular disease, impairment of consciousness, ataxia, seizures, neuralgia, skeletal muscle injury, corticospinal tract signs, meningitis, encephalitis, loss of smell and encephalopathy.

Disseminated intravascular coagulation is a condition in which small blood clots develop throughout the bloodstream, blocking small blood vessels. The increased clotting can deplete the platelets and clotting factors needed to control bleeding, and can cause excessive bleeding.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. As used in the specification and claims, the singular form “a”, “an” and “the” include plural references unless the context clearly dictates otherwise.

As used herein “immunization” is the process whereby individuals from a subpopulation are made immune or resistant to a disease caused by a pathogen, such as a virus or a bacterium. If, at least one month after the process of immunization, a group of immunized individuals is challenged with the pathogen against it was immunized, the group of immunized individuals will have lower mortality and/or lower morbidity than a comparable control group of unimmunized individuals challenged with the pathogen under comparable experimental conditions. The “immunized” individuals have gained “immunity”, a lower risk of mortality and/or a lower risk of morbidity compared to comparable individuals who are not immune.

As used herein an “agonist” is a chemical that binds to a receptor and activates the receptor to produce a biological response.

As used herein “epidemic” and “pandemic” adhere to the nomenclature of the Center for Disease Control, USA.

As used herein the term “prophylactic treatment” refers to the prevention of the onset, recurrence or spread of a disease or disorder, or of one or more symptoms thereof.

As used herein the term “infection fatality rate” is ratio between events of death among infected people and the total number of infected people.

As used herein “respiratory disease” is a disease of the respiratory tract, typically causing symptoms like difficult breathing, cough, breathing noisily, lingering chest pain and/or coughing up blood.

As used herein “tlr3” is the protein encoded by the human tlr3-gene. The tlr3 gene has the reference sequence ID NG_007278.

As used herein “tlr4” is the protein encoded by the human tlr4-gene. The tlr4 gene has the reference sequence ID NG_011475.

As used herein “tlr7” is the protein encoded by the human tlr7-gene. The tlr7 gene has the reference sequence ID NG_012569.

As used herein “tlr8” is the protein encoded by the human tlr8-gene. The tlr8 gene has the reference sequence ID NG_012882.

As used herein “tlr9” is the protein encoded by the human tlr9-gene. The tlr9 gene has the reference sequence ID NG_033933.

As used herein, the term “proximity” relates to nearness in space, such as being in the same house, on the same farm or in the same hospital.

As used herein, the term “dose” is a quantity of a drug to be taken at a particular time.

As used herein the term “symptomatic” relates to exhibiting characteristics of a particular disease.

As used herein the term “nonsymptomatic” or “asymptomatic” relates to not or not yet exhibiting the characteristics of a particular disease.

As used herein, “at least one” means “one or more.” As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human.

The terms “subject” or “patient” do not denote a particular age or sex. Thus, adult, infant and newborn subjects, whether male or female, are intended to be covered.

The term “SARS-COV-2” is used herein to define an RNA viral species

The term “emergency situation” as used herein is the immediate risk to contract the infectious agent causing the infection.

During the priority year several results were published by others which fit with this invention's underlying rationale for the prophylaxis and treatment of COVID-19, which are
development of severe infection as a result of an insufficient innate immune response due to delayed/subdued tlr7-signalling pathway at the very early stage of the infection, and
an overactive tlr7-signalling pathway as one driver for the “cytokine storm” observed at later stages of a severe infection.

With regard to the first part, van der Made et al. (2020) “Presence of genetic variants among young men with severe COVID-19.” JAMA 324:663-673 identified two sets of two young brothers (median age of 26) carrying a rare TLR7 loss of function variant who suffered from severe COVID-19. Fallerini C et al. (2021) “Association of Toll-like receptor 7 variants with life-threatening COVID-19 disease in males: findings from a nested case control study.” eLife 10:e67569, reported five cases of men (three under 50, and two in their mid-60s) with severe COVID-19 who carry rare TLR7 loss-of-function variants.

Based on these publications van de Veerdonk and Netea, eLife 2021; 10:e67860, conclude “We are therefore more confident with suggesting that variants of this single gene are responsible for an important proportion of risk factor for severe COVID-19 in men under 50. Functional studies have started to shed light on the mechanism by which TLR7 variants can lead to severe COVID-19. This work shows that the variants disrupt the production of type I and type Il interferon after stimulation of the TLR7 receptor, which suggests that the mutations lead to a loss-of-function in the antiviral response to SARS-COV-2.”The dependence on strong tlr7-signalling at the early stage of the infection might be seen as surprising at first sight, since there are TWO major pathways for the activation of type I IFNs following RNA virus infection: the RIG-I-like receptors (RLRs) and the Toll-like receptors (TLRs). Chen et al. “SARS-COV-2 Nucleocapsid Protein Interacts with RIG-I and Represses RIG-Mediated IFN-β Production.” Viruses 2021, 13, 47, reported that the SARS-COV-2 N protein represses IFN-β production by interfering with RIG-I. Zhen et al. “Severe acute respiratory syndrome coronavirus 2(SARSCOV-2 ) membrane (M) protein inhibits type I and III interferon production by targeting RIG-I/MDA-5 signaling” in Signal Transduction and Targeted Therapy (2020) 5:299, reported that the SARS-COV-2 M protein antagonizes type I and III IFN production by targeting RIG-I/MDA-5 signaling” In other word, the inhibition of the RIG-I pathway offers an explanation why the initial innate immune response is so very dependent on functional tlr7-signalling in the case of SARS-COV-2 infection.

A model emerges that upon viral entry into epithelial cells of the nasal cavity, interferon production by the infected cells is inhibited by viral proteins like the nucleocapsid protein and/or the M-protein and an early protective IFN-I response by the host becomes dependent on tlr-7-mediated sensing of viral RNA in plasmacytoid dendritic cells. If that response is missing—due to genetic defects, for example as described in the young and healthy, but vulnerable men identified by van der Made—or blunted—due to established tlr-tolerance in at-risk patent groups like the obese and elderly—then a SARS-COV-2 infection establishes itself and progresses to a dangerous stage. All of this highlights the importance of an early and healthy tlr7-response immediately upon infection and further strengthens the rationale for the use of a tlr agonist in the one-time prophylactic treatment against a coronavirus infection in order to protect a subpopulation of asymptomatic at-risk subjects in proximity to subjects of the population who show symptoms of a SARS-COV-2 infection.

With regard to the second part-overactive and detrimental tlr7-signalling at later stages of COVID, with symptoms similar to a tlr7-driven SLE-flare-further evidence has been published. Bowles et al. “Lupus Anticoagulant and Abnormal Coagulation Tests in Patients with Covid-19” N Engl J Med 2020; 383:288-290, found lupus anticoagulant in 91% of a cohort of patients with severe COVID 19. Vlachoyiannopoulos et al. “Autoantibodies related to systemic autoimmune rheumatic diseases in severely ill patients with COVID-19” in Ann Rheum Dis December 2020 Vol 79 No 1 found that 68.7% of a random cohort of severely ill Covid patients were positive for a systemic autoantibody, such as the antinuclear antibodies frequently observed in SLE-patients. Since the role of overactive tlr7-signalling in the development of these autoantibodies in SLE-patients is known, the appearance of these autoantibodies in patients with a severe form of Covid points to overactive tlr7-signalling in Covid patients at a later stage when the disease has progressed to a severe form.

Zhou et al. “Heightened Innate Immune Responses in the Respiratory Tract of COVID-19 Patients” Cell Host & Microbe 27, 883-890, found a robust IFN-response in the broncheoalveolar lavage of severe COVID-19 patients and an increase in activated dendritic cells, also indicative of active tlr7-signalling at that stage. Shaath et al. (2020) “Single-Cell Transcriptome Analysis Highlights a Role for Neutrophils and Inflammatory Macrophages in the Pathogenesis of Severe COVID-19” in Cells 2020, 9, 2374, PMID: 33138195, have compared gene signatures obtained by single cell sequencing of cells from bronchoalveolar lavage from patients with severe Covid with those of healthy patients. They observed an upregulation of the tlr7-transcript in patients with severe Covid and a downregulation of miR-146, which is a negative regulator of tlr7-signalling (FIG. 6b in Shaath). This offers a direct mechanistical basis for overactive tlr7-signalling in patients with severe Covid 19. Assuming that this upregulation of tlr7 and downregulation of miR-146 happened after infection in these patients—since a strong early tlr7-response would have protected them from the development of severe disease—this observation provides a rationale for how “resensitization” of tlr7-signalling happens after the viral infection has establish itself.

All of this highlights the problems that overactive tlr7-signalling plays once a severe form of the disease has developed and further strengthens the rationale for the use of an antagonist of tlr7-signalling in the treatment of severe COVID-19 wherein the antagonist of tlr7-signalling is to be administered after seroconversion.

The invention is further exemplified by the following embodiments
1. A method of providing immunity against a virus to a selected subpopulation of a larger population by
a) administering an agonist of a toll-like-receptor to individuals from the selected subpopulation, and then
b) exposing the individuals from step a) to the virus.
2. The method of embodiment 1, where step b) is at most seven days after step a).
3. The method of embodiment 2, where step b) is done from 3 days to immediately after step a).
4. The method of any one of embodiments 1 to 3, wherein the population is a human population.
5. The method of any one of embodiments 1 to 3, wherein the population is a population of mammalian animals.
6. The method of embodiments 4 or 5, wherein the subpopulation has an infection fatality rate that is known to be or expected to be below the average infection fatality rate of the population.
7. The method of embodiment 4 or 5, wherein the subpopulation is at an increased risk of exposure to the virus.
8. The method of embodiment 7 in combination with embodiment 4, wherein the subpopulation is medical personnel.
9. The method of any one of embodiments 1 to 8, wherein the virus causes a respiratory disease.
10. The method of any one of embodiments 1 to 9, wherein the virus is an RNA-virus.
11. The method of embodiment 10, wherein the RNA-virus is a coronavirus.
12. The method of embodiment 11, wherein the coronavirus is a SARS-like virus.
13. The method of embodiment 12, wherein the SARS-like virus is SARS-COV-2.
14. The method of any one of embodiments 1-13, wherein exposure to the virus in step b) and administration of the toll-like-receptor agonist in step a) are effected to the same part of the body.
15. The method of embodiment 14, wherein the part of the body is the upper respiratory tract.
16. The method of embodiment 15, wherein the part of the body is the nasal cavity.
17. The method of embodiment 5, wherein the virus causes a respiratory disease.
18. The method of embodiment 17, wherein the virus is an RNA-virus.
19. The method according to any one of embodiments 1 to 18, wherein the toll-like receptor is signalling via MyD88 or a mammalian homologue thereof.
20. The method of any one of embodiments 1 to 18, wherein the population is a human population and therein the toll-like receptor is tlr3, tlr4, tlr7, tlr8 and/or tlr9.
21. The method of any one of embodiments 1 to 20, wherein the tlr agonist is a tlr 3 agonist, a tlr4 agonist, a tlr7 agonist, a tlr8 agonist and/or a tlr9 agonist.
22. The method of any one of embodiments 1 to 21, wherein the tlr agonist is Poly(I:C), R848, imiquimod, VTX-2337, GLA-SE, SD-101, GSK2245035, RO7119929 or GSK1795091, in particular wherein the tlr-agonist is poly(I:C) and wherein from 1 mg to 20 mg poly(I:C) are to be administered nasally.
23. Use of a toll-like receptor agonist in the immunization of a subpopulation of a population during a virus outbreak.
24. The use according to embodiment 23, wherein the virus outbreak is a virus epidemic.
25. The use according to embodiments 23 and/or 24, wherein the virus outbreak is a pandemic.
26. The use according to any one of embodiments 23 to 25, wherein the toll-like receptor agonist is to be administered to a subpopulation who is at risk of being exposed to the virus within seven days after administration of the toll-like receptor agonist.
27. The use according to any one of embodiments 23 to 26, wherein the tlr agonist is as defined in any one of embodiments 21 or 22.
28. The use according to any one of embodiments 23 to 27, wherein the virus is as defined in any one of embodiments 9 to 13.
29. The use according to any one of embodiments 23 to 28, wherein the population is a human population.
30. The use according to embodiment 29, wherein the subpopulation has an infection fatality rate that is known to be or expected to be below the average infection fatality rate of the population.
31. The use according to embodiment 30, wherein the subpopulation is at an increased risk of exposure to the virus.
32. The use according to embodiment 31, wherein the subpopulation is medical personnel.
33. The use according to any one of embodiments 23 to 28, wherein the population is a mammalian animal population.
34. The use according to embodiment 33, wherein the subpopulation is at an increased risk of exposure to the virus.
35. The use according to embodiment 34, wherein the subpopulation at an increased risk of exposure to the virus is a collection of asymptomatic animals in proximity to animals who show symptoms of virus infection.
36. A single dose of a tlr agonist for the prophylactic treatment of a virus infection in a subpopulation, wherein the subpopulation is a collection of asymptomatic subjects in proximity to subjects of the population who show symptoms of virus infection.
37. The single dose according to embodiment 36, wherein the population is a human population.
38. The single dose according to embodiments 35 and 36, wherein the subpopulation is medical personnel of a hospital receiving subjects who show symptoms of virus infection.
39. The single dose according to embodiments 35 and 36, wherein the subpopulation has an infection fatality rate that is known to be or expected to be above the average infection fatality rate of the population and wherein.
40. The single dose according to embodiment 36, wherein the population is a mammalian animal population.
41. The single dose according to embodiment 40, wherein the mammalian animals are farm animals.
42. The single dose according to embodiment 41, wherein the farm animals are pigs, cows, sheep, goats or horses.
43. The single dose according to embodiment 42 wherein the farm animals are pigs and wherein the virus is a flavivirus or a asfarvirus.
44. A tlr agonist for use in the one-time prophylactic treatment of a subpopulation against an infection, wherein the subpopulation is a collection of asymptomatic subjects in proximity to subjects of the population who show symptoms of infection.
45. The tlr agonist for use in the one-time prophylactic treatment of embodiment 44, wherein the infection is a virus infection.
46. The tlr agonist for use in the one-time prophylactic treatment according to embodiments 44 or 45, wherein the population is a human population.
47. The tlr agonist for use in the one-time prophylactic treatment according to any one of embodiments 44 to 46, wherein the subpopulation is medical personnel of a hospital about to receive patients who show symptoms of virus infection.
48. The tlr agonist for use in the one-time prophylactic treatment according to any one of embodiments 44 to 46, wherein the subpopulation has an infection fatality rate that is known to be or expected to be above the average infection fatality rate of the population and wherein.
49. The tlr agonist for use in the one-time prophylactic treatment according to embodiments 44 or 45, wherein the population is a mammalian animal population except rodents.
50. The tlr agonist for use in the one-time prophylactic treatment according to embodiment 49, wherein the mammalian animals are farm animals.
51. The tlr agonist for use in the one-time prophylactic treatment according to embodiment 50, wherein the farm animals are pigs, cows, sheep, goats or horses.
52. The tlr agonist for use in the one-time prophylactic treatment according to embodiment 51 wherein the farm animals are pigs and wherein the virus is a flavivirus or an asfarvirus.
53. The tlr agonist for use in the one-time prophylactic treatment according to any one of embodiments 44 to 52 wherein the tlr agonist is a tlr 3 agonist, a tlr 4 agonist, a tlr7 agonist, a tlr8 agonist and/or a tlr9 agonist.
54. The tlr agonist for use in the one-time prophylactic treatment according to any one of embodiments 44 to 53 wherein the tlr agonist is a tlr 3 agonist, in particular wherein the tlr-agonist is poly(I:C) and wherein from 1 mg to 20 mg poly(I:C) are to be administered nasally.
55. The tlr agonist for use in the one-time prophylactic treatment according to any one of embodiments 44 to 53 wherein the tlr agonist is a tlr 4 agonist.
56. The tlr agonist for use in the one-time prophylactic treatment according to any one of embodiments 44 to 53 wherein the tlr agonist is a tlr7 agonist.
57. The tlr agonist for use in the one-time prophylactic treatment according to any one of embodiments 44 to 53 wherein the tlr agonist is a tlr 8 agonist.
58. The tlr agonist for use in the one-time prophylactic treatment according to any one of embodiments 44 to 53 wherein the tlr agonist is a tlr 9 agonist.
59. The tlr agonist for use in the one-time prophylactic treatment according to any one of embodiments 44 to 53 wherein the tlr agonist is Poly(I:C), resiquimod, imiquimod, VTX-2337, GLA-SE, SD-101, GSK2245035, RO7119929 or GSK1795091, in particular wherein the tlr-agonist is poly(I:C) and wherein from 1 mg to 20 mg poly(I:C) are to be administered nasally.
60. A tlr agonist for use in the prophylactic treatment of asymptomatic subjects against an infection in an emergency situation.
61. The tlr agonist for use according to embodiment 60, wherein the infection is a virus infection.
62. The tlr agonist for use according to embodiments 60 or 61, wherein the asymptomatic subject is a human being.
63. The tlr agonist for use according to any one of embodiments 60 to 62, wherein the human being is a member of medical personnel of a hospital and wherein the emergency situation is receiving a patient into the hospital who shows symptoms of virus infection.
64. The tlr agonist for use according to any one of embodiments 60 to 63, wherein the asymptomatic subject belongs to a subpopulation having an infection fatality rate that is known to be or expected to be above the average infection fatality rate of the population.
65. The tlr agonist for use according to embodiments 60 or 61, wherein the subject is a mammalian animal, preferably wherein the subject is not a rodent.
66. The tlr agonist for use according to embodiment 65, wherein the mammalian animal is a farm animal.
67. The tlr agonist for use according to embodiment 66, wherein the subject is a pig, a cow, a sheep, a goat or a horse.
68. The tlr agonist for use according to embodiment 67 wherein the subject is a pig and wherein the virus is a flavivirus or an asfarvirus.
69. The tlr agonist for use according to any one of embodiments 60 to 68 wherein the tlr agonist is a tlr3 agonist, a tlr4 agonist, a tlr7 agonist, a tlr8 agonist and/or a tlr9 agonist.
70. The tlr agonist for use according to any one of embodiments 60 to 69 wherein the tlr agonist is a tlr3 agonist, in particular wherein the tlr-agonist is poly(I:C) and wherein from 1 mg to 20 mg poly(I:C) are to be administered nasally.
71. The tlr agonist for use according to any one of embodiments 60 to 69 wherein the tlr agonist is a tlr4 agonist.
72. The tlr agonist for use according to any one of embodiments 60 to 69 wherein the tlr agonist is a tlr7 agonist.
73. The tlr agonist for use according to any one of embodiments 60 to 69 wherein the tlr agonist is a tlr8 agonist.
74. The tlr agonist for use according to any one of embodiments 60 to 69 wherein the tlr agonist is a tlr9 agonist.
75. The tlr agonist for use according to any one of embodiments 60 to 69 wherein the tlr agonist is Poly(I:C), resiquimod, imiquimod, VTX-2337, GLA-SE, SD-101, GSK2245035, RO7119929 or GSK1795091, in particular wherein the tlr-agonist is poly(I:C) and wherein from 1 mg to 20 mg poly(I:C) are to be administered nasally.
76. The tlr agonist for use according to any one of embodiments 60 to 75, wherein the emergency situation is the immediate risk to contract the infectious agent causative for the infection.
77. The tlr agonist for use according to any one of embodiments 60 to 75, wherein the emergency situation is the proximity of subjects who show symptoms of infection.
78. The tlr agonist for use according to any one of embodiments 60 to 75, wherein the emergency situation is a newly detected outbreak of the infection in a building where more than one subject lives or works and wherein the subjects are human being.
79. The tlr agonist for use according to any one of embodiments 60 to 75, wherein the emergency situation is a newly detected outbreak of the infection in a building, where subjects live or work who are expected to have an infection fatality rate that is known to be or expected to be above the average infection fatality rate of the population.
80. The tlr agonist for use according to any one of embodiments 60 to 75, wherein the emergency situation is a newly detected outbreak of a virus infection in an old people's care facility and wherein the subjects are human being.
81. The tlr agonist for use according to any one of embodiments 60 to 75, wherein the emergency situation is a newly detected outbreak of a virus infection on a farm and wherein the subjects are farm animals.
82. The tlr agonist for use according to any one of embodiments 60 to 75, wherein the emergency situation is a newly detected outbreak of a swine virus infection on a farm and wherein the subjects are pigs.
83. The tlr agonist for use according to any one of embodiments 60 to 75, wherein the emergency situation is a newly detected outbreak of a swine fever virus infection on a farm, wherein the subjects are pigs and wherein the virus is a flavivirus or an asfarvirus.
84. An antagonist of tlr7-signalling for use in the treatment of pulmonary inflammation resulting from viral infection.
85. The antagonist of tlr7-signalling for use according to embodiment 84, wherein the antagonist of tlr7-signalling is to be administered after viral clearance.
86. The antagonist of tlr7-signalling for use according to any one of embodiments 84 or 85, wherein the viral infection is an infection with a coronavirus.
87. The antagonist of tlr7-signalling for use according to embodiment 86, wherein the coronavirus is a SARS-like coronavirus, in particular wherein the coronavirus is SARS-COV-2.
88. The antagonist of tlr7-signalling for use according to any one of embodiments 84 to 87, wherein the antagonist is chloroquine, hydroxychloroquine, quinacrine or vitamin D.
89. The antagonist of tlr7-signalling for use according to any one of embodiments 84 to 88, wherein viral clearance has been determined by a PCR- or RT-PCR based assay.
90. The antagonist of tlr7-signalling for use according to embodiment 89, wherein viral clearance has been determined by at least to negative results in PCR- or RT-PCR analysis of patient samples.
91. The antagonist of tlr7-signalling for use according to embodiment 90, wherein the patient samples have been taken from locations in the body where the virus is known to persist the longest.
92. The antagonist of tlr7-signalling for use according to embodiment 91, wherein the patient samples taken for PCR- or RT-PCR analysis have been taken from at least two independent locations.
93. An antagonist of tlr7-signalling for use in the treatment of thrombosis resulting from viral infection, wherein the antagonist of tlr7-signalling is to be administered after viral clearance.
94. The antagonist of tlr7-signalling for use according to embodiment 93, wherein the virus is a coronavirus, in particular a SARS-like coronavirus.
95. The antagonist of tlr7-signalling for use according to embodiment 94, wherein the virus is SARS-COV-2.
96. An antagonist of tlr7-signalling for use in the prevention of lung embolism resulting from viral infection, wherein the antagonist of tlr7-signalling is to be administered after viral clearance.
97. The antagonist of tlr7-signalling for use according to embodiment 96, wherein the virus is a coronavirus, in particular a SARS-like coronavirus.
98. The antagonist of tlr7-signalling for use according to embodiment 97, wherein the virus is SARS-COV-2.
99. An antagonist of tlr7-signalling for use in the treatment of a disease caused by coronavirus infection, such as COVID-19, wherein the antagonist of tlr7-signalling is to be administered after seroconversion.
100. The antagonist of tlr7-signalling for use according to embodiment 99, wherein the antagonist is chloroquine, hydroxychloroquine, quinacrine or vitamin D.
101. The antagonist of tlr7-signalling for use according to any one of embodiments 99 to 100, wherein seroconversion has been determined by an FDA-approved antibody assay.
102. The antagonist of tlr7-signalling for use according to embodiment 101, wherein seroconversion has been determined by at least two independent measurements.
103. The antagonist of tlr7-signalling for use according to any one of embodiments 99 to 102, wherein the subject suffering from COVID-19 infection has an age of at least 70 years.
104. The antagonist of tlr7-signalling for use according to any one of embodiments 99 to 103, wherein the subject suffering from COVID-19 infection has a body mass index of at least 30.
105. The antagonist of tlr7-signalling for use according to any one of embodiments 99 to 104, wherein the subject suffering from COVID-19 infection shows at least one complication selected from the group consisting of venous thromboembolism, kidney injury, cytokine release syndrome, neurological symptoms and disseminated intravascular coagulation.
106. The antagonist of tlr7-signalling for use according to any one of embodiments 99 to 105, wherein the antagonist of tlr7-signalling is not to be administered to a subject suffering from a disease caused by a coronavirus infection, such as COVID-19, prior to seroconversion.
107. The antagonist of tlr7-signalling for use according to any one of embodiments 99 to 105, wherein no antagonist of tlr7-signalling has been administered to a subject suffering from a disease caused by a coronavirus infection, such as COVID-19, prior to seroconversion.
108. The antagonist of tlr7-signalling for use according to any one of embodiments 106 or 107, wherein anti-inflammatory drugs are not to be administered to a subject suffering from a disease caused by a coronavirus infection, such as COVID-19, prior to seroconversion.
109. The antagonist of tlr7-signalling for use according to according to any one of embodiments 106 or 107, wherein nonsteroidal anti-inflammatory drugs are not to be administered to a subject suffering from a disease caused by a coronavirus infection, such as COVID-19, prior to seroconversion.
110. The antagonist of tlr7-signalling for use according to any one of embodiments 106 or 107, wherein steroidal anti-inflammatory drugs, for example prednisone, cortisone or methylprednisone, are not to be administered to a subject suffering from a disease caused by a coronavirus infection, such as COVID-19, prior to seroconversion.
111. The antagonist of tlr7-signalling for use according to any one of embodiments 99 to 110, wherein seroconversion is determined by the appearance of coronavirus-specific IgG antibodies.
112. The antagonist of tlr7-signalling for use according to embodiment 111, wherein the coronavirus is SARS-COV-2 and wherein the coronavirus-specific IgG antibodies are SARS-CoV-2 -specific IgG antibodies.
113. A drug, which drug is safe and efficacous for the treatment of systemic lupus erythematosus, for use in the treatment of a disease caused by coronavirus infection, such as COVID-19, wherein the drug is to be administered after seroconversion.
114. The drug for use according to embodiment 113, wherein the coronavirus is a SARS-like coronavirus.
115. The drug for use according to embodiment 113, wherein the coronavirus is SARS-COV-2.
116. The drug for use according to any one of embodiments 113 to 115, wherein seroconversion has been determined by an FDA-approved antibody assay.
117. The drug for use according to embodiment 116, wherein seroconversion has been determined by at least two independent measurements.
118. The drug for use according to any one of embodiments 113 to 117, wherein the subject suffering from the coronavirus infection has an age of at least 70 years.
119. The drug for use according to any one of embodiments 113 to 118, wherein the subject suffering from the coronavirus infection has a body mass index of at least 30.
120. The drug for use according to any one of embodiments 113 to 119, wherein the subject suffering from coronavirus infection shows at least one complication selected from the group consisting of venous thromboembolism, kidney injury, cytokine release syndrome, neurological symptoms and disseminated intravascular coagulation.
121. The drug for use according to any one of embodiments 113 to 120, wherein said drug is not to be administered to a subject suffering from the disease caused by a coronavirus infection, such as COVID-19, prior to seroconversion.
122. The drug for use according to any one of embodiments 113 to 121, wherein said drug has not been administered to a subject suffering from the disease caused by a coronavirus infection, such as COVID-19, prior to seroconversion.
123. The drug for use according to any one of embodiments 113 to 122, wherein anti-inflammatory drugs are not to be administered to the subject suffering from the disease caused by a coronavirus infection, such as COVID-19, prior to seroconversion.
124. The drug for use according to any one of embodiments 121 or 122, wherein nonsteroidal anti-inflammatory drugs are not to be administered to the subject suffering from the disease caused by a coronavirus infection, such as COVID-19, prior to seroconversion.
125. The drug for use according to any one of embodiments 121 or 122, wherein steroidal anti-inflammatory drugs, for example prednisone, cortisone or methylprednisone, are not to be administered to the subject suffering from the disease caused by a coronavirus infection, such as COVID-19, prior to seroconversion.
126. The drug for use according to any one of embodiments 113 to 125, wherein seroconversion is determined by the appearance of coronavirus-specific IgG antibodies.
127. The drug for use according to embodiment 126, wherein the coronavirus is SARS-COV-2 and wherein the coronavirus-specific IgG antibodies are SARS-COV-2-specific IgG antibodies.
128. A treatment regimen, which treatment regimen is safe and efficacous for the treatment of systemic lupus erythematosus, for use in the treatment of a disease caused by coronavirus infection, such as COVID-19, wherein the drug is to be administered after seroconversion.
129. The treatment regimen for use according to embodiment 128, wherein the coronavirus is a SARS-like coronavirus.
130. The treatment regimen for use according to embodiment 128, wherein the coronavirus is SARS-COV-2.
131. The treatment regimen for use according to any one of embodiments 128 to 130, wherein seroconversion is to be determined by an FDA-approved antibody assay.
132. The treatment regimen for use according to embodiment 131, wherein seroconversion is to be determined by at least two independent measurements.
133. The treatment regimen for use according to any one of embodiments 128 to 132, wherein the subject suffering from the coronavirus infection has an age of at least 70 years.
134. The treatment regimen for use according to any one of embodiments 128 to 133, wherein the subject suffering from the coronavirus infection has a body mass index of at least 30.
135. The treatment regimen for use according to any one of embodiments 128 to 134, wherein the subject suffering from the coronavirus infection shows at least one complication selected from the group consisting of venous thromboembolism, kidney injury, cytokine release syndrome, neurological symptoms and disseminated intravascular coagulation.
136. The treatment regimen for use according to any one of embodiments 128 to 135, wherein said treatment regimen is not to be applied to said subject suffering from a disease caused by a coronavirus infection, such as COVID-19, prior to seroconversion.
137. The treatment regimen for use according to any one of embodiments 128 to 136, wherein said treatment regimen has not been applied to the subject suffering from a disease caused by a coronavirus infection, such as COVID-19, prior to seroconversion.
138. The treatment regimen for use according to any one of embodiments 128 to 137, wherein anti-inflammatory drugs are not to be administered to the subject suffering from the disease caused by a coronavirus infection, such as COVID-19, prior to seroconversion.
139. The treatment regimen for use according to any one of embodiments 136 or 137, wherein nonsteroidal anti-inflammatory drugs are not to be administered to the subject suffering from the disease caused by a coronavirus infection, such as COVID-19, prior to seroconversion.
140. The treatment regimen for use according to any one of embodiments 136 or 137, wherein steroidal anti-inflammatory drugs, for example prednisone, cortisone or methylprednisone, are not to be administered to the subject suffering from the disease caused by a coronavirus infection, such as COVID-19, prior to seroconversion.
141. The treatment regimen for use according to any one of embodiments 128 to 140, wherein seroconversion is determined by the appearance of coronavirus-specific IgG antibodies.
142. The treatment regimen for use according to embodiment 141, wherein the coronavirus is SARS-COV-2 and wherein the coronavirus-specific IgG antibodies are SARS-COV-2-specific IgG antibodies.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Example 1

The experiment shown in FIG. 1A and C of Zhao et al., J. Virol. 2012 November, 86(21):11416-11424 is repeated. B6 mice are lightly anesthetized with isoflurane and infected intranasally with 1×105 PFU of SARS-COV. Aged mice are infected with 105 PFU SARS-COV-1 . Mice are first treated with agonists for TLR3 (poly(I·C), TLR4 (LPS), TLR7/8 (resiquimod), and TLR9 (CpG) and then infected with SARS-COV-1 shortly after pretreatment. Treatment with agonists for TRL3, TLR7/8, and TLR9 all increase survival, pretreatment with poly(I·C) is most protective.

After 10 to 14 days mice show complete virus clearance. After 14 more days, the surviving mice are investigated for the presence of neutralizing antibodys for SARS-COV-1.

Several mice have developed neutralizing antibodies against the SARS-COV-1 virus.

### Example 2

21 days after complete virus clearance 10 mice which have survived the first challenge with SARS-COV-1 are again infected with 105 PFU SARS-COV-1 . More than one mouse survives this second challenge, indicating that mice which survive the first SARS-COV-1 challenge with the help of the pretreatment with a tlr agonist develop some immunity against the virus.

### Example 3

In a farm where a first case of classical swine fever is reported among a population of at least 100 pigs, all but one symptomatic pigs are culled. 30 apparently unsymptomatic pigs are treated intranasally with 0.1 mg/kg poly(I:C) and marked so that these pretreated individuals can be identified. One hour after the pretreatment the whole population of apparently unsymptomatic pigs, which includes at least 30 unsymptomatic pigs which have not received a pretreatment, is allowed to mingle with the infected pig for 24 hours. The infected pig is then culled and removed and the pretreated pigs are then separated from the rest of the population. Pigs who develop symptoms of severe classical swine fever are culled and counted as dead. The infection fatality rate is determined for the subpopulation of poly(I:C)-pretreated pigs and for the subpopulation which did not receive a pretreatment. The infection fatality rate for the subpopulation of pretreated pigs is lower than the infection fatality rate of the untreated subpopulation.

### Example 4

In an old people's home where the first patient with COVID-19 symptoms is detected, to half of the still asymptomatic inhabitants (number of all inhabitants is at least 20) of age 65 or higher 6.4 mg (4×800 μg per nostril using a dry powder applicator) of the tlr 3 agonist PrEP-001 (contains poly(I:C)) are administered once, similar to what has been described in detail in the context of a clinical trial in Malcom et al., Antiviral Res. 2018 May; 153:70-77. The other half of the still asymptomatic patients receive placebo dry powder. After three weeks, there are fewer deaths and/or fewer severe cases requiring intensive care in the treatment group vs. the placebo group.

### Example 5

12 MRL/MpJ-Faslpr (MRL-Faslpr) mice are challenged with a non-lethal dose of SARS-CoV-2 at age 10-12 weeks, an age where autoimmune MRL-Faslpr mice have developed autoantibodies and glomerulonephritis. 6C 57BL/6 (B6) mice are included for comparison as a non-autoimmune model. After viral clearance, starting around day 15 after virus infection, 6 of the 12 MRL-Faslpr mice are given an intraperitoneal injection of 40 mg/kg/day hydroxychloroquine.

Pulmonary pathology is inspected at day 40 post infection. Mice are sacrificed and lungs are inflated and fixed with 10% neutral buffered formalin (Sigma-Aldrich) and embedded in paraffin. Lung sections (5 μM thickness) are stained with hematoxylin and eosin, as described in detail in Slight-Webb SR et al., J Autoimmun. 2015 February; 57:66-76. The severity of pulmonary inflammation is blindly scored on a scale of 0-4, with a score of 4 being the most severe. The severe inflammation visible in untreated MRL-Faslpr mice is improved by the hydroxychloroquine treatment.

1.-28. (canceled)

29. An antagonist of tlr7-signalling for use in the treatment of COVID-19, wherein the antagonist of tlr7-signalling is to be administered after seroconversion.

30. The antagonist of tlr7-signalling for use according to claim 29, wherein seroconversion has been determined by an FDA-approved antibody assay.

31. The antagonists of tlr7-signalling for use according to claim 29, wherein the subject suffering from COVID-19 infection has an age of at least 70 years.

32. The antagonists of tlr7-signalling for use according to claim 29, wherein the subject suffering from COVID-19 infection has a body mass index of at least 30.

33. The antagonists of tlr7-signalling for use according to claim 29, wherein the subject suffering from COVID-19 infection shows at least one complication selected from the group consisting of venous thromboembolism, kidney injury, cytokine release syndrome, neurological symptoms and disseminated intravascular coagulation.

34. The antagonists of tlr7-signalling for use according to claim 29, wherein the antagonists of tlr7-signalling is not to be administered to a subject suffering from COVID-19 prior to seroconversion.

35. The antagonists of tlr7-signalling for use according to claim 29, wherein no antagonist of tlr7-signalling has been administered to a subject suffering from COVID-19 prior to seroconversion.

36. The antagonists of tlr7-signalling for use according to claim 29, wherein anti-inflammatory drugs are not to be administered to a subject suffering from COVID-19 prior to seroconversion.

37. The antagonists of tlr7-signalling for use according to claim 29, wherein nonsteroidal anti-inflammatory drugs are not to be administered to a subject suffering from COVID-19 prior to seroconversion.

38. The antagonists of tlr7-signalling for use according to claim 29, wherein the antagonist of tlr7-signalling is a drug which is safe and efficacous for the treatment of systemic lupus erythematosus.

39. A tlr agonist for use in the one-time prophylactic treatment of a subpopulation against an infection with a coronavirus, wherein the subpopulation is a collection of asymptomatic subjects in proximity to subjects of the population who show symptoms of infection.

40. The tlr agonist for use in the one-time prophylactic treatment according to claim 39, wherein the subpopulation has an infection fatality rate that is known to be or expected to be above the average infection fatality rate of the population.

41. The tlr agonist for use in the one-time prophylactic treatment according to claim 39 wherein the tlr agonist is a tlr3 agonist, a tlr4 agonist, a tlr7 agonist, a tlrS agonist and/or a tlr9 agonist.

42. The tlr agonist for use in the one-time prophylactic treatment according to claim 39 wherein the tlr agonist is a tlr3 agonist, in particular wherein the tlr-agonist is poly(I:C) and wherein from 1 mg to 20 mg poly(I:C) are to be administered nasally.

43. A tlr agonist for use in the prophylactic treatment of asymptomatic subjects against an infection with a coronavirus in an emergency situation.

44. The tlr agonist for use according to claim 43, wherein the asymptomatic subject belongs to a subpopulation having an infection fatality rate that is known to be or expected to be above the average infection fatality rate of the population.

45. The tlr agonist for use according to claim 43 wherein the tlr agonist is a tlr3 agonist, a tlr4 agonist, a tlr7 agonist, a tlr8 agonist and/or a tlr9 agonist.

46. The tlr agonist for use according to claim 45 wherein the tlr agonist is a tlr3 agonist, in particular wherein the tlr-agonist is poly(I:C) and wherein from 1 mg to 20 mg poly(I:C) are to be administered nasally.

47. The tlr agonist for use according to claim 43 wherein the emergency situation is the immediate risk to contract the infectious agent causative for the infection.

48. The tlr agonist for use according to claim 43, wherein the emergency situation is the proximity of subjects who show symptoms of infection.
